(19) United States
(12) Patent Application Publication
(10) Pub. No.: US 2014/0213980 A1
(43) Pub. Date: Jul. 31, 2014

(54) Medical cuff

(71) Applicant: Deana M. Filippelli, Warwick, RI (US)

(72) Inventor: Deana M. Filippelli, Warwick, RI (US)

(21) Appl. No.: 13/815,043

(22) Filed: Jan. 25, 2013

(51) Int. Cl. A61M 25/02

(52) U.S. Cl. CPC A61M 25/02
USPC 604/179

(57) ABSTRACT

A wrist-worn device having a tether forming a bridge under which medical tubing can be extended and adapted to be worn by a patient undergoing fluid infusion via an IV insertion point so that the tubing, when subjected to accidental pulling or tugging, is restrained by the tether from damaging the IV connection point.

This invention claims the benefit of U.S. Provisional Patent Application Ser. No. 61/633,435 filed Feb. 10, 2012 entitled MEDICAL CUFF BY DEANA M. FILIPPELLI.

### BACKGROUND OF THE INVENTION

This invention pertains to a medical cuff and more specifically to an encircling band adapted to be worn on the arm of a medical patent. The band includes a tether-like portion under which a medical tube is loosely passed such that movement of the patient's arm will not cause the tubing to harmfully pull on the IV line, PICC or other intravenous portal to which the tubing is connected.

Medical tubing used within a hospital/surgical setting is normally left unattached after the insertion point has been created. The IV insertion point or portal is normally sited on the inner forearm of the patient. Thereafter when infusion of fluids into a patient via the insertion point is desired, tubing is reconnected to the insertion point. Infusion can take place for significant periods of time dependent upon the patient's illness or medical needs, e.g., chemotherapy. During and between infusions, the tubes are left hanging or dangling freely from the insertion point or secured to the patient's body in a primitive manner such as by applying surgical tape. These tubes left unsecured can become tangled around body parts, e.g., arms or legs, can be stepped on especially by young or elderly patients or become obstructed by patients confined to bed. Any of these foregoing situations can cause the point of insertion either via IV tube or intravenous portal to become dislodged resulting in discomfort and distress to patients. In many such incidents, the portals require examination by staff and potentially re-accessed ultimately resulting in additional cost outlays to the medical facility.

The device of the present invention provides a “holder” or cuff that is both reusable and comfortable. The design of the present invention allows the medical tubes to be securely fastened while still passing freely beneath a bridge formed by the attached tether. The effective length of the tether is adjustable therefore allowing for more than one tube to be concurrently fastened beneath the bridge. The device of the present invention is meant to be worn on the patient's wrist but can also be worn on a patient's arm, leg or ankle. Since the device of the present invention gathers tubes in one central location, the tubes are less likely to become tangled or accidentally caught or wedged on an object. Because the device is attached around the patient's wrist, arm, leg or ankle, natural patient movement is also achieved.

Other objects, features and advantages of the invention shall become apparent as the description thereof proceeds when considered in connection with the accompanying illustrative drawings.

### SUMMARY OF THE INVENTION

The holder is comprised of two main components: the base component is a band preferably constructed of a reusable and washable material having elasticity providing for wearing comfort, and the second component is the tether also preferably constructed of a washable material and secured with dual snaps or other closure means such as hook and loop tape. This tether is inserted through slits, holes or openings incorporated within the band or cuff. The tether is secured to the band via dual snaps. This allows the tubes to be inserted into the holder “post” medical connection of the tube(s) to the point of IV insertion. Because the tether incorporates fastening elements, e.g., snap closures, the tubing can be easily placed under the tether. By design, the invention can allow for small, medium, and large tethers that will accommodate a single to several tubes simultaneously. The tether will also allow for various tube sizes. The band's elasticity accommodates patients of all shapes and sizes.

### DESCRIPTION OF THE DRAWINGS

In the drawings which illustrate the best mode presently contemplated for carrying out the present invention:

FIG. 1 is a top stylized view of one form of the device of the present invention;

FIG. 2 is a partial cross-sectional view taken along the line 2-2 of FIG. 1;

FIG. 3 is a top plan view similar to FIG. 1 with one portion of the tether unfastened to show how the bridge over the upper surface of the band is formed;

FIG. 4 is a top stylized view of the device of the present invention being worn by a patient;

FIG. 5 is a top stylized view of an alternate form of the device of the present invention; and

FIG. 6 is a top stylized view of still another alternate form of the invention.

### DESCRIPTION OF THE INVENTION

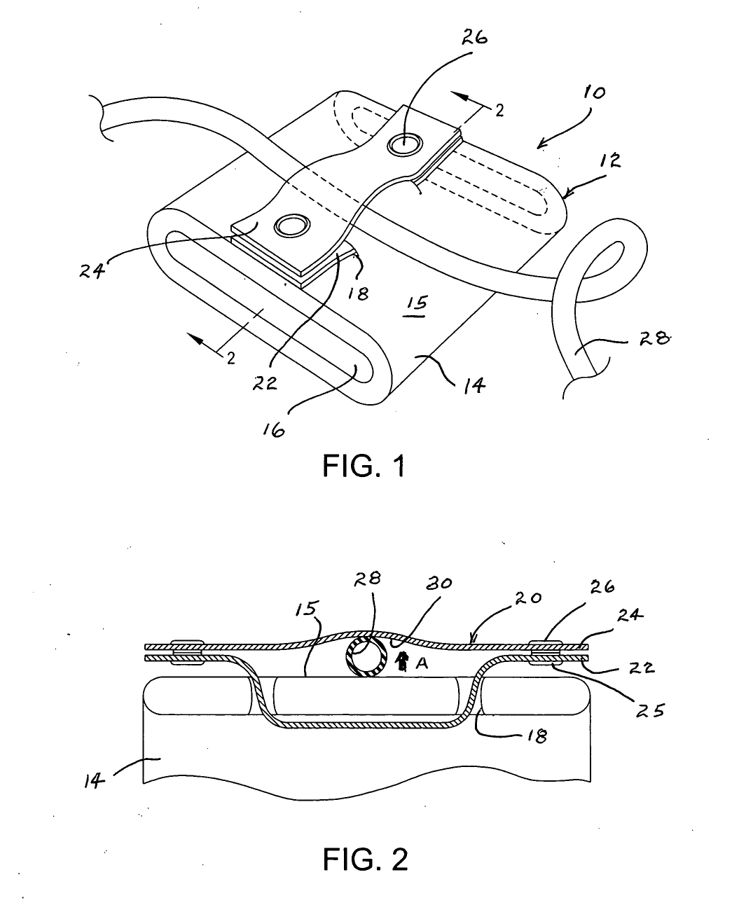

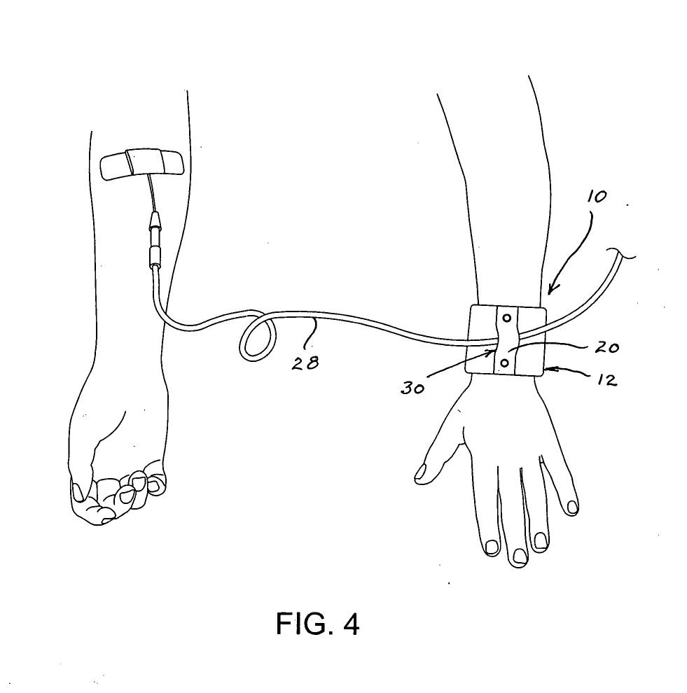

Referring to the drawings and particularly FIGS. 1-4, the device 10 includes band 12 adapted to encircle the patient's appropriate body part (usually the wrist) and includes a generally cylindrical body 14 having an upper surface 15 with open ends 16 and a pair of slits or openings 18 in the upper surface 15 thereof. The slits 18 laterally extend a short distance and are longitudinally spaced from each other and serve to receive the lower strip 22 of an adjustable tether 20. The tether 20 is preferably formed of a washable fabric and preferably of a different color from that of the band 12 to more clearly differentiate between the band 12 and the tether 20.

The tether 20 also includes an upper strip 24 similar to strip 22 and both strips 22, 24 include longitudinally spaced snap fasteners 25, 26 respectively such that when the lower strip 22 is inserted though the slits 18 and the upper strip 24 placed thereover, the snap fasteners 25, 26 can be aligned and when connected to each other, in effect, form a bridge 30 over the band upper surface 15 under or through which the medical tubing 28 may be positioned. Alternatively, the strips 22, 24 may be formed of one integral strip threaded through the slits 18 and then folded over itself to achieve a similar effect. The snap fasteners 25, 26 can be conventional metal snaps used in the garment industry or formed of a non-metallic composition such as plastic, nylon or polypropylene to eliminate any hospital concern relative to a patient wearing the device when undergoing a MRI or other metal sensitive testing procedures.

Thus, the medical tubing 28 is free to move laterally through the tunnel or bridge 30 formed by the lower surface of the upper strip 24 and the s upper surface 15 of the band 12 when the tubing 28 is inadvertently pulled or tugged upon yet is restrained from upwardly, that is, movement outwardly from the band upper surface, by the upper strip (see in particular FIG. 2 which is a section through line 2-2 of FIG. 1 and the directional arrow A representing such undesirable tubing movement). The bridge 30 also provides longitudinal movement restraint for the tubing 28 such that the tubing 28, when subject to longitudinally directed force with respect to the band 12, will contact one of the snap connections 25, 26 on either side of the tunnel or bridge. Generally, the pulling or tugging to which the tubing is accidentally or purposely subjected includes both lateral and longitudinal force components. Accordingly, the device 10 is constructed to generally permit lateral movement of the tubing 28 especially when purposely directed through the bridge 30 as well as restraining longitudinal and upward movement. Such restraint helps prevent undesirable tugging on the IV attachment site.

More than two equidistantly spaced snaps may be placed on the strips 22, 24 such that the longitudinal spacing of the snaps can be varied to accommodate variously sized and quantity of medical tubing. Also, different strips with differently spaced snaps can be utilized to achieve the above adjustability result.

In addition, snaps may be affixed directly to the band 12 such as when the band 12 is constructed of a relatively durable material, e.g., the elastic materials used in wrist braces; and in such cases, the lower strip 22 would be eliminated and the upper strip 24 and the snaps thereon attached directly to the band. It should also be pointed out that the snaps are in reality a specific form of attachment or connection means and other connection means may be substituted for the snaps. For instance, hook and loop strips such as Velcro may be utilized instead of snaps.

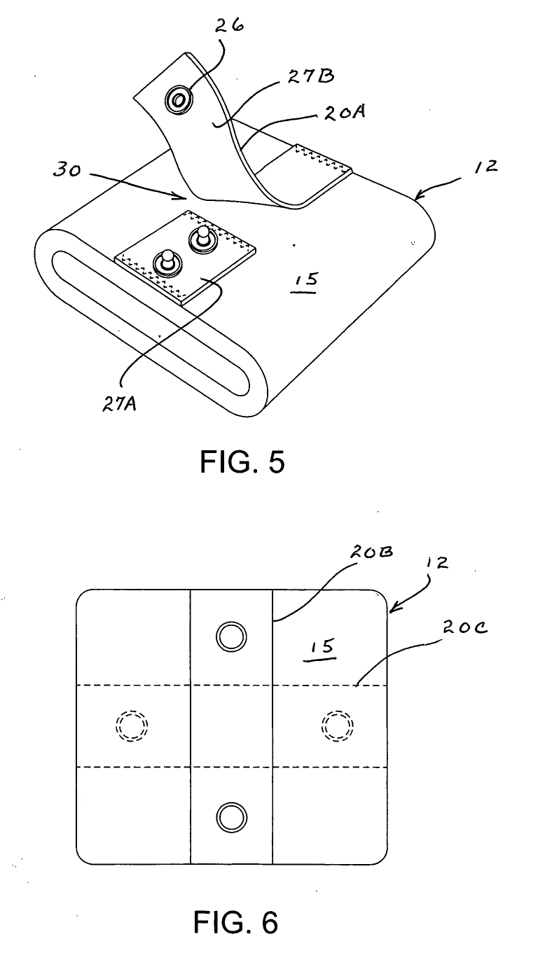

Referring to FIG. 5, an alternate form of the invention is shown. Therein, the band 12 includes only a two-component upper strip 27A, 27B that replaces strip 24 in the previously described embodiments. Both strips 27A, 27B are directly attached to the upper surface 15 of the band 12 as by sewing. The strip 27A may also include two or more snaps longitudinally spaced from each other to enable a bridge formation of various sizes to accommodate different tubing and/or place greater restraint on tubing movement.

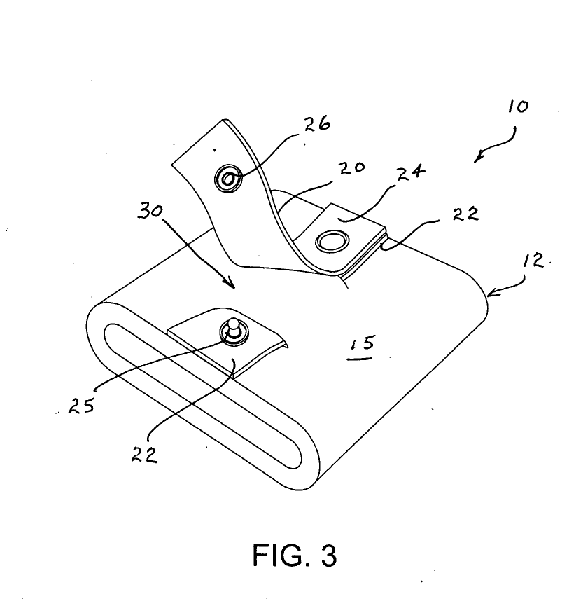

Referring to FIG. 6, a still further modification of the device of the present invention is shown. Therein, two tethers 20 are positioned on opposite surfaces of the band 12 and oriented in normal, i.e., 90°, opposition to each other such that the band 12 may be worn so as to position either of such tethers 20B,20C in the upward tube engaging position to enable primary tube restraint in either the longitudinal or lateral direction. The tethers depicted in FIG. 6 can be of either of the configurations shown above and referred to as reference numeral 20 in FIG. 3 and reference numeral 20A in FIG. 5.

While there is shown and described herein certain specific structure embodying this invention, it will be manifest to those skilled in the art that various modifications and rearrangements of the parts may be made without to departing from the spirit and scope of the underlying inventive concept and that the same is not limited to the particular forms herein shown and described except insofar as indicated by the scope of the appended claims.

What is claimed is:

1. A medical cuff adapted for positioning upon an arm of a patient having an IV insertion point positioned thereon for restraining undesirable movement of medical tubing connected to the IV insertion point, said cuff comprising an arm positioning band having opposed upper and lower surfaces and a tether anchored on said band upper surface, said tether comprising at least one elongated strip having opposed ends connected to said cuff via fastening elements incorporated into said strip at said opposed ends thereof, said strip positioned on said cuff so as to extend longitudinally thereacross and having a lower surface positioned above said upper cuff surface so as to form a bridge above said cuff upper surface, said bridge being intermediate of said fastening elements and wherein medical tubing is adapted for laterally extending positioning beneath said bridge whereupon the tubing is relatively free to move laterally with respect to the cuff but is simultaneously restrained from both longitudinal and upward movement with respect to said cuff.

2. The medical cuff of claim 1, said cuff upper surface includes a pair of longitudinally spaced slits, said strip extending downwardly through one of said slits and then beneath the upper cuff surface and then upwardly outwardly through the other of said slits and then folded over itself, said strip having at least two sets of fastening elements which when interconnected form said bridge.

3. The medical cuff of claim 2, said fastening elements being snap fasteners.

4. The medical cuff of claim 2, said strip being of two-part construction wherein one part thereof extends beneath said cuff upper surface via said slits and the other part is positioned over said one strip part to form said bridge.

5. The medical cuff of claim 1, wherein said strip is of two-part construction and wherein one part thereof includes a free end and an opposite end directly attached to said cuff and a second part connected to said cuff and positioned longitudinally spaced from said one part, both said parts including fastening elements.

6. The medical cuff of claim 5, wherein said second part of said strip includes plural longitudinally spaced fastening elements so as to enable a varied height bridge configuration.

7. The medical cuff of claim 1, wherein said band is adapted to encircle the arm and is of generally toroidal configuration.

8. The medical cuff of claim 1, wherein said band includes an additional tether positioned on the lower cuff surface, said additional tether including a bridge-forming strip positioned normal to said strip positioned on the band upper surface.

9. In combination with a medical installation of an IV insertion point system positioned on one arm of a patient wherein medical tubing is connected to said insertion point system and extends between said insertion point and a source of fluid for infusion into the patient, a medical cuff adapted for positioning upon the opposite arm of the patient, said cuff comprising an arm positioning band having opposed upper and lower surfaces and a tether anchored on said band upper surface, said tether comprising at least one elongated strip having opposed ends connected to said cuff via fastening elements incorporated into said strip at said opposed ends thereof, said strip positioned on said cuff so as to extend longitudinally thereacross and having a lower surface positioned above said upper cuff surface so as to form a bridge above said cuff upper surface, said bridge being intermediate of said fastening elements and wherein medical tubing is adapted for laterally extending positioning beneath said bridge whereupon the tubing is relatively free to move laterally with respect to the cuff but is simultaneously restrained from both longitudinal and upward movement with respect to said cuff.

10. The combination set forth in claim 9, wherein said medical cuff is adapted to encircle said opposite arm and is of generally toroidal configuration.
